(19) United States
(12) Patent Application Publication
(10) Pub. No.: US 2002/0128176 A1
(43) Pub. Date: Sep. 12, 2002

(54) Treatment of erectile dysfunctions with C-Type natriuretic polypeptide (CNP) as a monotherapy or in combination with phosphodiesterase inhibitors

(76) Inventors: Wolf-Georg Forssmann, Hannover (DE); Hans-Jurgen Magert, Hannover (DE); Christian Stief, Hemmingen (DE); Andrea Kuthe, Hannover (DE); Stefan Uckert, Garbsen (DE); Armin Becker, Hannover (DE); Udo Jonas, Hannover (DE)

Correspondence Address: JACOBSON HOLMAN, PROFESSIONAL LIMITED LIABILITY COMPANY, 400 SEVENTH STREET, N.W., WASHINGTON, DC, 20004, US

(21) Appl. No.: 09/912,425

(22) Filed: Jul. 26, 2001

Related U.S. Application Data
(63) Continuation of application No. PCT/EP00/00634, filed on Jan. 27, 2000.

(30) Foreign Application Priority Data
Jan. 27, 1999 (DE) 199 03 087.1

(51) Int. Cl. A61K 38/17

(52) U.S. Cl. 514/2

(57) ABSTRACT

A medicament containing a combination of CNP or fragments or derivatives thereof with retained physiological activity of CNP and phosphodiesterase inhibitors.

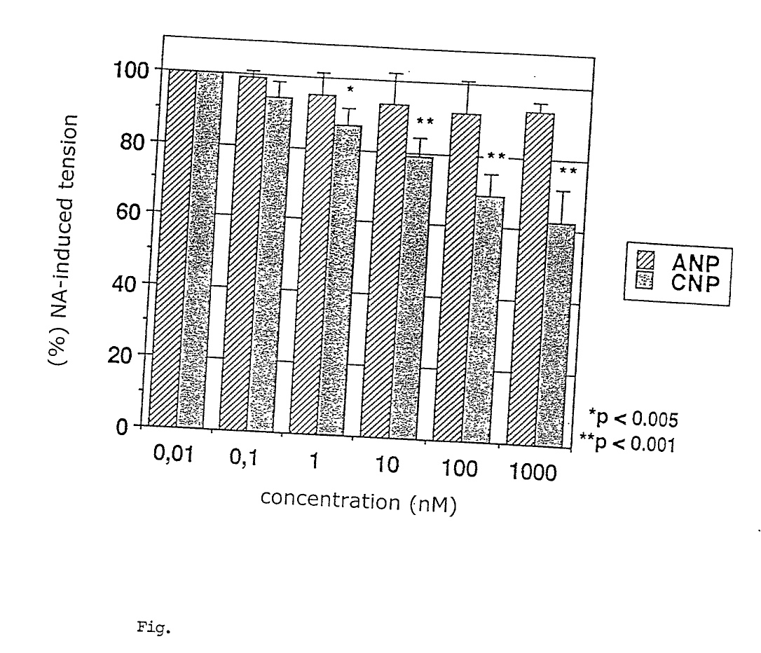

The present invention relates to a medicament for the treatment of erectile dysfunctions, and the use of C-type natriuretic polypeptide (CNP) and its variants singly or in combination with phosphodiesterase inhibitors for the treatment of erectile dysfunctions.

The relaxation of the smooth muscles of the corpus cavernosum penis, which leads to penile erection, is induced by a complex interplay of different factors, the ultimately formed intracellular secondary messenger cyclic adenosine monophosphate (cAMP) playing an essential role (Stief et al., 1997). Its concentration is determined, on the one hand, by the activity of cAMP-synthesis catalyzing enzymes, namely adenylate cyclases, and on the other hand, by cAMP-degrading (hydrolyzing) enzymes, the phosphodiesterases (PDEs). To date, more than ten different phosphodiesterase isoenzymes are known, some of which hydrolyze cAMP, but others of which also hydrolyze the related secondary messenger cyclic guanosine monophosphate (cGMP) (Beavo and Reifsnyder, 1990; Burns et al., 1996; Taher et al., 1997). Since a cAMP-hydrolyzing phosphodiesterase, PDE3A, is inhibited by cGMP, the cGMP-hydrolyzing phosphodiesterase 5 (PDE5) has itself come into the focus of the research into erectile dysfunction in recent years. Sildenafil is a PDE5 inhibitor which is currently being successfully employed under the designation Viagra as a therapeutic for the treatment of erectile dysfunctions (Boolell et al., 1997). However, undesirable side-effects of Sildenafil, such as flickering in the eyes, headaches or circulatory failure, have been described.

It has been the object of the present invention to provide an agent which reduces or avoids the mentioned side-effects.

This object is achieved by a medicament containing a combination of CNP and phosphodiesterase inhibitors. CNP may also be replaced or supplemented by fragments or derivatives thereof with retained physiological activity of CNP. Useful fragments include, in particular, subfragments contained in the CNP prohormone sequence. The amino acid sequences of CNP and of proCNP and of preproCNP are shown in the following:

Human preproCNP (Prepropeptide)

MHLSQLLACALLLTLLSLRPSEAKPGAPPKVPRTPPAEELAEPQAAGGG
QKKGDKAPGGGGANLKGDRSRLLRDLRVDTKSRAAWARLLQEHPNAR
KYKGANKKGLSKGCFGLKLDRIGSMSGLGC

Human proCNP (Propeptide)

KPGAPPKVPRTPPAEELAEPQAAGGGQKKGDKAPGGGGANLKGDRSR
LLRDLRVDTKSRAAWARLLQEHPNARKYKGANKKGLSKGCFGLKLDRI
GSMSGLGC

Human CNP (Mature Peptide)

GLSKGCFGLKLDRIGSMSGLGC

As derivatives, there may be used, in particular, those structures derived from the formula shown which are produced by the conservative substitution of amino acids in this sequence. Further, amidated, phosphorylated, acylated, alkylated derivatives and/or derivatives of CNP containing linkages between side chains may be used.

The deletion of amino acids not involved in the function also yields fragments which may be employed as a medicament according to the invention.

Preferably, the medicament further contains necessary auxiliaries and/or carriers which enable or support the galenic application. For the intravenous administration of the medicament according to the invention, it is recommendable to dissolve the respective components in appropriate formulations suitable for intravenous administration. Further application forms include oral, intracavernous, intraurethral, locally topical, transcutaneous.

As the phosphodiesterase inhibitors, there may be used, in particular, those of PDE5 phosphodiesterase. This include, in particular, Zaprinast, Dipyridamole, Milrinone and/or Sildenafil.

According to the invention, the use of CNP or fragments or derivatives of CNP with retained physiological activity of CNP is claimed for the treatment of erectile dysfunctions. In an advantageous embodiment of the use according to the invention, CNP or fragments or derivatives of CNP with retained physiological activity of CNP are employed in combination with phosphodiesterase inhibitors.

According to the invention, an increase of the intracellular cAMP level is achieved by the stimulation of the formation (not inhibition of hydrolysis) of the PDE3A inhibitor cGMP.

In tests made with male patients, both the intravenous and the intracavernous application of CNP resulted in an immediate erection which was maintained for about one hour. Surprisingly, in the combined application with phosphodiesterase inhibitors, such as Sildenafil, it was found that their necessary doses for the induction of an erection could be significantly decreased by the simultaneous application of CNP. In some subjects, the side-effects encountered in the exclusive Sildenafil treatment, such as flickering in the eyes or headaches, were also reduced, in part to complete disappearance.

The Figure shows the effect of CNP and ANP/CDD₉₉₋₁₂₆ on preparations of smooth muscle of the human corpus cavernosum penis in the form of the effects of CNP and ANP/CDD₉₋₁₂₆ on the relaxation of precontracted preparations of smooth muscle. In contrast to ANP/CDD₉₉₋₁₂₆, CNP exhibits a significant relaxant effect already at a concentration of 1 nM, which eventually leads to a relaxation corresponding to 60% of the tonus found upon norepinephrine administration when the concentration is increased to 1 μM.

### EXAMPLE 1

Among the enzymes catalyzing the synthesis of cGMP, the guanylate cyclases, it can be distinguished between two different subgroups, namely the soluble cytosolic guanylate cyclases, and the membrane-bound ones (for a survey, see Wedel and Garbers, 1997). To date, it has been considered that the soluble cytosolic guanylate cyclases played an exclusive role in the induction of erection. To examine a possible participation of membrane-bound guanylate cyclases, in a first step, the expression of the genes for the membrane-bound guanylate cyclases GC-A, GC-B and GC-C and for two retinal membrane-bound guanylate cyclases (data base identifiers: HSRETGC and HSRETCG) in the corpus cavernosum tissue was checked as follows using the RT-PCR technique:

The total RNA was isolated from biopsy samples of human corpus cavernosum, followed by transcription of the messenger RNA (mRNA) contained in 5 μg of total RNA into copy DNA primary strand (cDNA primary strand) as described previously (Mägert et al., 1995; Mägert et al., 1998). 1/300 each of the cDNA primary strand samples was subsequently employed in standard RT-PCR reactions (Saiki et al., 1988) with primer pairs specific for the various guanylate cyclases as derived from cDNA data base entries. The reaction conditions were as follows:

| | | | |
|---|---|---|---|
| | 1 × denaturing | 94° C. | 5 min |
| followed by | 35 × each denaturing | 98° C. | 1 sec |
| | primer hybridization | 50° C. | 30 sec |
| | elongation | 72° C. | 1 min |
| finally | 1 x elongation | 72° C. | 7 min |

The products obtained were separated by electrophoresis on an agarose gel and visualized by means of ultraviolet transmitted light upon ethidium bromide staining.

Actually, with the primers specific for guanylate cyclase B (GC-B) (sense primer GCB-1S: GGGTGCTGTGGCCTCTGGTTTTTCGG, antisense primer GCB-2AS: CCCTGCATCTTTTCCACAATTCGAAG), a homogeneous band could be obtained within the proper size range. The isolation of the corresponding DNA fragment from the agarose gel, the subsequent sequencing with a DNA fluorescence sequencer using the primers employed for amplification, and the comparison of the sequence obtained with the entries in the EMBL/GenBank data bases finally confirmed it as being GC-B specific.

### EXAMPLE 2

Since it is known that GC-B represents the receptor for the peptide hormone C-type natriuretic polypeptide (CNP) produced in the brain, it was examined in the following way whether human CNP has relaxing effects on the smooth muscle of the corpus cavernosum or induces an increase of the intracellular concentration of cyclic nucleoside monophosphates:

Functional organ bath studies:

Strip preparations of human cavernous muscles were fixed in the bath chambers of an organ bath plant (Isolated Organ Apparatus IOA 5306, Föhr Medical Instruments GmbH, Seeheim, Germany) filled with a modified Krebs-Ringer solution (NaCl 120 mM, NaHCO₃ 25.6 mM, KCl 4.7 mM, CaCl₂ 2.5 mM, NaH₂PO₄ 1.2 mM, MgCl₂ 1.2 mM, glucose 22 mM, 2Na⁺ (Ca²⁺) EDTA 0.1 mM, pH 7.2-7.4). The temperature of the bath solution was controlled to 37° C., and a mixture of 950/% O₂ and 5% CO₂ was continuously sparkled through the solution. The application of a passive bias tension of 5 mN (0.5 g) was followed by an equilibration phase of 60 min. Subsequently, the muscle strips were stimulated with 6 μM norepinephrine (NOR). After a stable contraction plateau was reached, the peptides human atrial natriuretic peptide/cardiodilatin (ANP/CDD₉₉₋₁₂₆) (for a survey, see Forssmann et al., 1998) and human C-type natriuretic peptide (CNP) (Ogawa et al., 1992), which are chemically synthesized in our laboratory, were added with cumulative dosage (0.01 to 1000 nM). Changes in the mechanical tension of the muscles were recorded with an analog-digital measured data acquisition system (MacLab®, AD Instruments, Castle Hill, Australia).

Determination of the Tissue Concentrations of Cyclic Nucleotides:

For determining time- and dose-dependent effects of ANP/CDD₉₉₋₁₂₆ and CNP on the intracellular concentration of cAMP and cGMP, strips of cavernous muscles were incubated with Krebs-Ringer solution in 2 ml reaction vessels by analogy with the organ bath experiments, continuously gassed with Carbogen, and stimulated with 6 μM NOR. This was followed by incubation with ANP/CDD₉₉₋₁₂₆ and CNP (0.1, 10 and 1000 nM) for 10 min. This was done in the presence of 1 mM isobutylmethylxanthin (IBMX) in order to prevent the degradation of cyclic nucleotides from the activity of phosphodiesterases. After the end of the incubation time, the tissue strips were transferred into prepared reaction vessels, shock-frozen in liquid nitrogen and subsequently homogenized to a fine powder. The extraction of cyclic nucleotides was effected by resuspending the tissue homogenizates in 1 ml of 7 00% ethanol, sonification of the suspensions for: 7 min, and finally centrifugation (10 min at 1,700 g). The alcoholic supernatants were withdrawn and freeze-dried, and the lyophilizates were taken up in 1 ml of RIA buffer (50 mM NaAc, pH 6.0). 200 μl each of the resuspended samples was charged into reaction vessels and acetylated with 6.7 p1 each of a mixture of triethylamine/acetic acid (ratio 2:1). The quantification of cAMP and cGMP was effected with radioimmunochemical methods using rabbit antibodies against acetylated cAMP and cGMP.

In the test series with different peptide concentrations, a significant relaxation could be achieved already from 1 nM CNP, which even corresponded to 600/% of the initial tension when the concentration was increased to 1 μM. With ANP/CDD₉₉₋₁₂₆ (the ligand of guanylate cyclase A), no significant relaxation was seen at corresponding concentrations, which confirms the importance of CNP and its receptor, guanylate cyclase B, to the tonus regulation of the penile smooth muscles (see Figure).

### REFERENCES

Beavo, J. A. and Reifsnyder, D. H. (1990). Primary sequence of cyclic nucleotide phosphodiesterase isoenzymes and the design of selective inhibitors. Trends Pharmac. Sci. 11: 150-155.

Boolell, M., Allen, M. J., Ballard, S. A., Gepi-Attee, S., Muirhead, G. J., Naylor, A. M., Osterloh, I. H. and Gingell, C. (1997). Sildenafil: an orally active type 5 cyclic GMP-specific phosphodiesterase inhibitor for the treatment of penile erectile dysfunction. Int. J. Impot. Res. 8: 47-52.

Burns, F., Zhao, A. Z. and Beavo, J. A. (1996). Cyclic nucleotide phosphodiesterases: gene complexity, regulation by phosphorylation, and physiological implications. Adv. Pharmacol. 36: 29-48.

Forssmann, W. G., Richter, R. and Meyer, M. (1998). The endocrine heart and natriuretic peptides: histochemistry, cell biology, and functional aspects of the renal urodilatin system. Histochem. Cell. Biol. 110: 335-357.

Magert, H. J., Reinecke, M., David, I., Raab, H. R., Adermann, K., Zucht, H. D., Hill, O., Hess, R. and Forssmann, W. G. (1998). Uroguanylin: Gene structure, expression, processing as a peptide hormone, and co-storage with somatostatin in gastrointestinal D-cells. Regul. Pept. 73: 165-176.

Magert, H. J., Hadrys, T., Cieslak, A., Groger, A., Feller, S. and Forssmann, W. G. (1995). cDNA sequence and expression pattern of the putative pheromone carrier aphrodisin. Proc. Natl. Acad. Sci. USA 92: 2091-2095.

Ogawa, Y., Nakao, K., Nakagawa, O., Komatsu, Y., Hosoda, K., Suga, S., Arai, H., Nagata, K., Yoshida, N. and Imura, H. (1992). Human C-type natriuretic peptide. Characterization of the gene and peptide. Hypertension 19: 809-813.

Saiki, R. K., Gelfand, D. H., Stoffel, S., Scharf, S. J., Higuchi, R., Horn, G. T., Mullis, K. B. and Ehrlich, H. A. (1998). Primer-directed enzymatic amplification of DNA with a thermostable DNA polymerase. Science 239: 487-491.

Stief, C. G., Noack, T. and Anderson, K. E. (1997). Signal transduction in cavernous smooth muscle. World ]. Urol. 15: 27-31.

Taher, A., Meyer, M., Stief, C. G., Jonas, U. and Forssmann, W. G. (1997). Cyclic nucleotide phosphodiesterase in human cavernous smooth muscle. World J. Urol. 15: 32-35.

Wedel, B. J. and Garbers, D. L. (1997). New insights on the functions of the guanylyl cyclase receptors. FEBS Lett. 410: 29-33.

1. A medicament containing a combination of CNP or fragments or derivatives thereof with retained physiological activity of CNP and phosphodiesterase inhibitors.

2. The medicament according to claim 1, further containing auxiliaries and/or carriers.

3. The medicament according to claim 2, wherein the medicament can be administered by intravenous, oral, intracavernous, intraurethral, locally topical or transcutaneous administration by means of said auxiliaries and/or carriers.

4. The medicament according to any of claims 1 to 3, wherein said phosphodiesterase inhibitors are Zaprinast, Dipyridamole, Milrinone and/or Sildenafil.

5. Use of CNP or fragments or derivatives of CNP with retained physiological activity of CNP for the treatment of erectile dysfunctions.

6. The use according to claim 5 in combination with phosphodiesterase inhibitors.


SEQUENCE LISTING (5 sequences)

SEQ ID NO: 1 - protein, 126 aa, Homo sapiens
MHLSQLLACALLLTLLSLRPSEAKPGAPPKVPRTPPAEELAEPQAAGGGQKKGDKAPGGGGANLKGDRSRLLRDLRVDTKSRAAWARLLQEHPNARKYKGANKKGLSKGCFGLKLDRIGSMSGLGC

SEQ ID NO: 2 - protein, 103 aa, Homo sapiens
KPGAPPKVPRTPPAEELAEPQAAGGGQKKGDKAPGGGGANLKGDRSRLLRDLRVDTKSRAAWARLLQEHPNARKYKGANKKGLSKGCFGLKLDRIGSMSGLGC

SEQ ID NO: 3 - protein, 22 aa, Homo sapiens
GLSKGCFGLKLDRIGSMSGLGC

SEQ ID NO: 4 - DNA, 26 bp, Artificial Sequence
  Description of Artificial Sequence PCR-Primer
gggtgctgtggcctctggtttttcgg

SEQ ID NO: 5 - DNA, 26 bp, Artificial Sequence
  Description of Artificial Sequence PCR-Primer
ccctgcatcttttccacaattcgaag